(19) United States
(12) Patent Application Publication
(10) Pub. No.: US 2019/0059698 A1
(43) Pub. Date: Feb. 28, 2019

(54) CABLE AND ENDOSCOPE USING THE SAME

(71) Applicant: OPCOM INC., New Taipei City (TW)

(72) Inventor: Chih-Yu Ting, New Taipei City (TW)

(21) Appl. No.: 16/028,428

(22) Filed: Jul. 6, 2018

Related U.S. Application Data
(60) Provisional application No. 62/551,274, filed on Aug. 29, 2017.

(51) Int. Cl. A61B 1/005; A61B 1/00; A61B 1/045

(52) U.S. Cl. CPC A61B 1/0052; A61B 1/05; A61B 1/045; A61B 1/00114

(57) ABSTRACT

A cable and an endoscope using the same are provided. A cable includes a housing, a circuit board, at least two buttons and a fixed portion. The housing has a space formed therein and a first opening connected to the space. The circuit board is received in the space and includes a first extending portion, a second extending portion and an included angle between the first extending portion and the second extending portion. The buttons are disposed in the first extending portion and the second extending portion, respectively. The fixed portion is disposed on each button and the first opening.

### CROSS REFERENCE TO RELATED APPLICATIONS

This application claims the benefit of U.S. Provisional Application No. 62/551,274, which was filed on Aug. 29, 2017, and is incorporated herein by reference.

### BACKGROUND OF THE INVENTION

### 1. Field of the Invention

The invention relates in general to a cable and an endoscope using the same, and more particularly to a cable with buttons and an endoscope using the same.

### 2. Description of the Prior Art

Currently, an operating element of an endoscope, such as button, is usually set or disposed on a handle of the endoscope. Although the handle has many functions, it also becomes very heavy, and it is very inconvenient to be hold or operated. And a user even may control the handle stably in accordance with both hands of the user.

Due to the handle is the most important component of the endoscope and controls almost elements of the endoscope, therefore, the using rate of the handle is very high, and the proportion of goods damaged and the using rate is in direct proportion. In addition, the button is disposed on the handle, and if the button is broke, and the broken button is not repaired, the handle must be replaced by a new one, even the endoscope may be replace by a new endoscope.

The handle caused by the broken button must be replace by a new one, and the cost of the endoscope is increase, and if the elements which is broke easily are all disposed on the handle, the attrition rate of the handle maybe increase and the attrition rate of the handle is never reduced, when the elements which is broke easily disposed on the handle.

### SUMMARY OF THE INVENTION

The disclosure is directed to a cable and an endoscope using the same. The cable has at least two buttons.

According to one aspect of the present disclosure, a cable is provided. A cable includes a housing, a circuit board, at least two buttons and a fixed portion. The housing has a space formed therein and a first opening connected to the space. The circuit board is received in the space and includes a first extending portion, a second extending portion and an included angle between the first extending portion and the second extending portion. The buttons are disposed in the first extending portion and the second extending portion, respectively. The fixed portion is disposed on each button and the first opening.

According to another aspect of the present disclosure, an endoscope is provided. An endoscope includes the cable described above, an insert tube and a handle. One end of the handle connects to one end of the cable, and the other end of the handle connects to one end of the insert tube.

According to another aspect of the present disclosure, an endoscope is provided. The endoscope includes an insert tube, a cable and a handle. The cable includes a housing, a circuit board, at least two buttons and a fixed portion. The housing has a space formed therein and a first opening connected to the space. The circuit board is received in the space and includes a first extending portion, a second extending portion and an included angle between the first extending portion and the second extending portion. The buttons are disposed in the first extending portion and the second extending portion, respectively. The fixed portion is disposed on each button and the first opening. One end of the handle connects to one end of the cable, and the other end of the handle connects to one end of the insert tube.

By the features described above, the present invention provides a cable and an endoscope using the same, the buttons are disposed on the cable, and the buttons not only controls the elements of the front end of the endoscope, such as, the camera module and the bending section of the insert tube but also operates the back end device connected to the cable. Due to the distance between the cable and the back end device is smaller than the distance between the handle and the back end device, therefore, an operating signal of the buttons of the cable may be transferred to the back end device fast and easily. When the buttons are broke, the broken cable may be replaced by a new cable to avoid repair the whole endoscope and achieve good reliability and durability.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1A is a side view schematically showing a whole endoscope according to one embodiment of the present invention.

FIG. 1B is an exploded view of a cable and a handle connected to an insert tube of the endoscope of FIG. 1A.

FIG. 2 is an exploded view schematically showing a cable according to one embodiment of the present invention.

### DETAILED DESCRIPTION

Preferred embodiments are disclosed below for elaborating the invention. Various lengths, such as optical focus lengths or distances, of a plurality of micro lenses are applied on a plurality of detecting pixels, such that in an autofocus process, the time consumption can be reduced, and the accuracy can be improved. The following embodiments are for the purpose of elaboration only, not for limiting the scope of protection of the invention. Besides, secondary elements are omitted in the following embodiments to highlight the technical features of the invention.

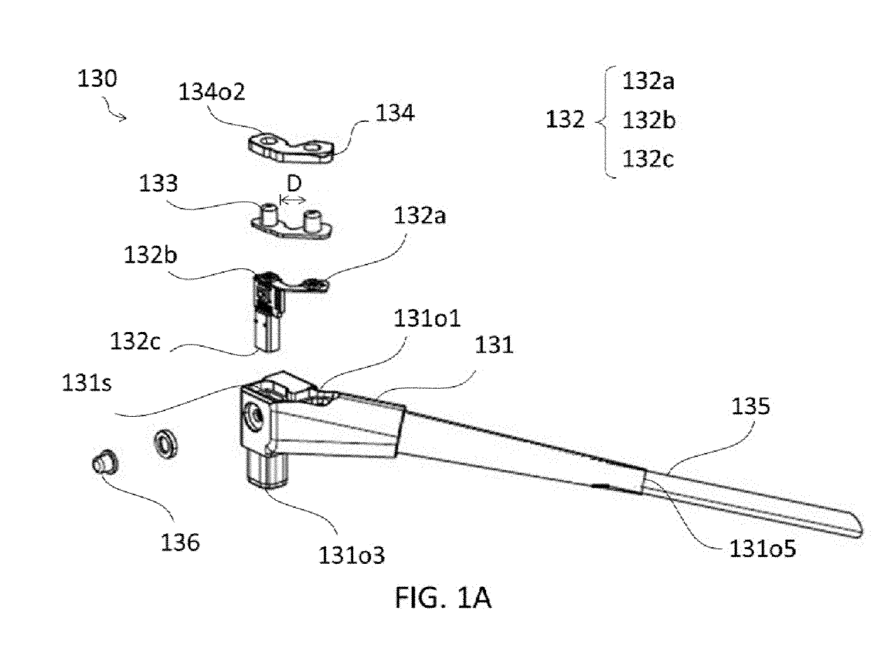

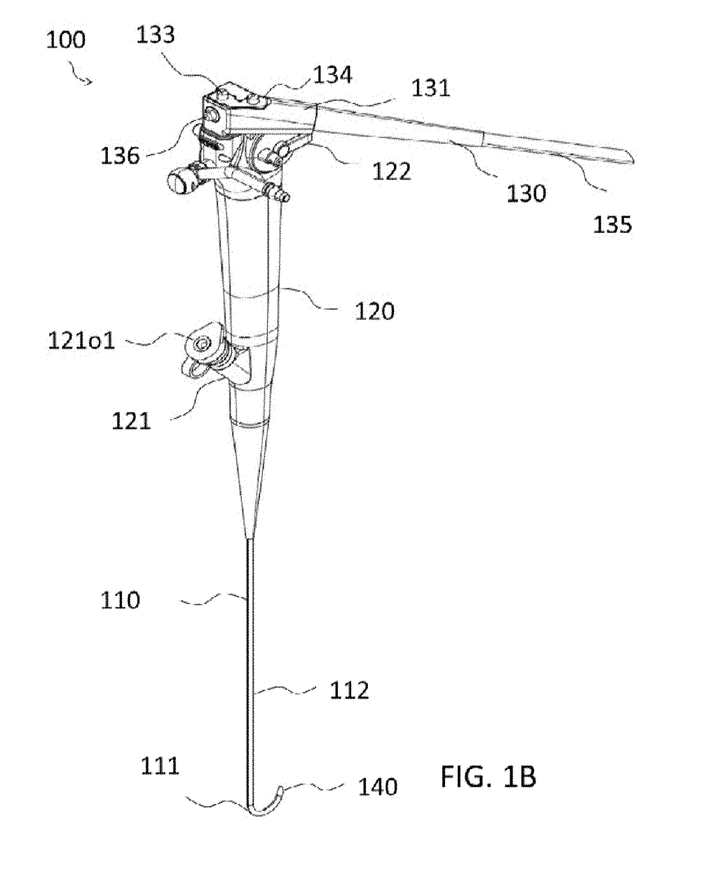

Reference is now made to FIG. 1A, a side view schematically showing a whole endoscope according to one embodiment of the present invention. Reference is now made to FIG. 1B, an exploded view of the cable and the handle connected to the insert tube of the endoscope of FIG. 1A. As shown in FIG. 1A and FIG. 1B, the endoscope 100 includes an insert tube 110, a handle 120, a cable 130 and a distal section 140. Wherein, one end of the handle 120 connects to one end of the cable 130, and the other end of the handle 120 connects to one end of the insert tube 110. The endoscope 100 may be a disposable endoscope or a reusable endoscope. The disposable endoscope or the reusable endoscope may be chosen in accordance with user's needed. The insert tube 110 may be a flexible tube or an inflexible tube. But it is not limit this invention.

As shown in FIG. 1B, the cable 130 and the handle 120 may be detached with each other. In the other words, the cable 130 and the handle 120 may be exploded with each other; therefore, the cable 130 may be replaced by another cable 130.

As shown in FIG. 1A and FIG. 1B, in an alternative embodiment, the cable 130 may be applied to an endoscope (no shown) . In other words, the cable 130 may be applied and/or configured another type of the endoscope with each other. Therefore, the cable 130 may replace the cable of another type of the endoscope.

In an alternative embodiment, the other end of the cable 130 may further connect to a back end device, such as, a main system (no shown), the main system includes at least one display, the display displays images captured from the endoscope 100.

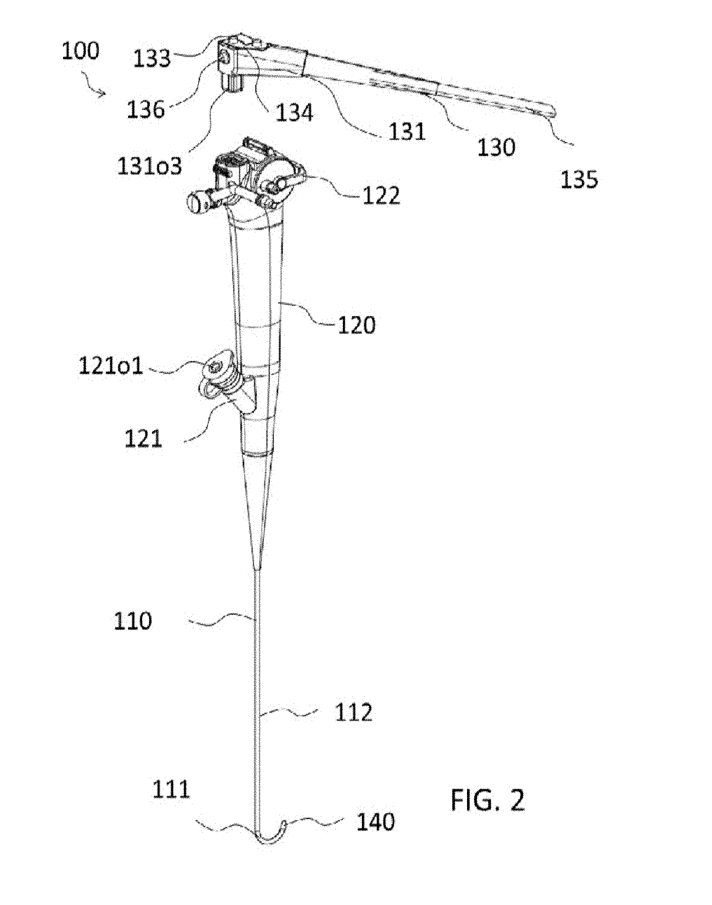

Reference is now made to FIG. 2, an exploded view schematically showing a cable according to one embodiment of the present invention. As shown in FIG. 1A, FIG. 1B and 2, in the current embodiment, the cable 130 includes a housing 131, a circuit board 132, at least two buttons 133, a fixed portion 134 and a wire 135. The housing 131 has a space 131s formed therein and a first opening 131o1 connected to the space 131s. The circuit board 132 is received in the space 131s, and the buttons 133 are disposed in the circuit board 132, respectively, the fixed portion 134 is disposed on each button 133 and also disposed the first opening 131o1.

Moreover, one part of the cable 130 has a space 131s formed therein, with this embodiments, the space 131s may be formed from a recess, and a shape of the space 131s may be designed in accordance with user's need, the elements received into the space 131s of the cable 130 as needed, or according to a shape of the circuit board 132 and the number of the buttons 133 disposed in the space 131s of the cable 130, but it is not limit this invention.

In another alternative embodiment, the housing 131 may further construct from four side faces and a bottom face (no shown), and four side faces connect with each other, and connect to the first opening 131o1 and a bottom face. The bottom face and the first opening 131o1 are corresponding with each other, and the space 131s are formed from four side faces and the bottom face.

In this embodiment, the circuit board 132 of the cable 130 includes a first extending portion 132a, a second extending portion 132b, a connected portion 132c and a transmitting line (no shown). An included angle is formed between the first extending portion 132a and the second extending portion 132b. In the embodiment, when the circuit board 132 is received in the space 131s, a face of the first extending portion 132a and a face of the second extending portion 132b are corresponding to the first opening 131o1 of the cable 130, respectively. The face of the first extending portion 132a and the face of the second extending portion 132b may be on the same face of on the different faces, but it is not limit this invention. The included angle between the first extending portion 132a and the second extending portion 132b of the circuit board 132 may be changed by different needs, and the size and/or the volume of the cable 130 may be changed in accordance with the needs. When the included angle between the first extending portion 132a and the second extending portion 132b of the circuit board 132 may be greater, the size and/or the cable 130 may be greater. When the included angle between the first extending portion 132a and the second extending portion 132b of the circuit board 132 is smaller, the size and/or the cable 130 maybe smaller. Therefore, for example, the minification of the whole cable 130 may be in accordance with the included angle between the first extending portion 132a and the second extending portion 132b of the circuit board 132 is smaller. In this embodiment, the included angle is greater than 0 degree and smaller than 180 degrees.

In one embodiment, a third opening 131o3 is formed on a face of one end of the housing 131 of the cable 130, and a fifth opening 131o5 is formed on a face of the other end of the housing 131, the face of the end of the housing 131 may be corresponding or not corresponding to the face of the other end of the housing 131, but it is not limit this invention. In other words, the third opening 131o3 is formed on the end of the cable 130, and the fifth opening 131o5 is formed on the other end of the cable 130. The connected portion 132c and the transmitting line (no shown) of the circuit board 132 are located at the third opening 131o3 and the fifth opening 131o5, respectively. In this embodiment, when the end and the other end of the cable 130 connect to the end of the handle 120 and a back end device, such as, a main system (no shown) , respectively, the cable 130 electronically connects to the handle 120 in accordance with the connected portion 132c of the circuit board 132 of the cable 130 via the third opening 131o3, and the cable 130 electronically connects to the back end device (no shown) in accordance with the transmitting line of the circuit board 132 of the cable 130 through the fifth opening 13105 and disposed through the wire 135.

As shown in FIG. 1A, FIG. 1B and 2, in the current embodiment, when the circuit board 132 is received and disposed in the space 131s of the housing 131, the circuit board 132 and the housing 131 may be connected and fixed with each other in accordance with the methods of engaging, locking or fixing. In this embodiment, for example, the circuit board 132 and the housing 131 may be engaged with each other in accordance with at least one engager 136, but it is not limit this invention.

In the current embodiment, for example, the cable 130 may include two buttons 133. The buttons 133 are disposed on the first extending portion 132a and the second extending portion 132b of the circuit board 132, respectively. Moreover, two buttons 133 are further disposed on the face of the first extending portion 132a and the face of the second extending 132b of the circuit board 132, respectively, and each button 133 is corresponding to the first opening 131o1 and exposed from the first opening 131o1. In this embodiment, two buttons 133 are neighbor with each other, a distance D may between two buttons 133. The included angle between the first extending portion 132a and the second extending portion 132b may be changed to change the distance D between two buttons. When the included angle between the first extending portion 132a and the second extending portion 132b is smaller, the buttons 133 are closer with each other; therefore, the distance D of buttons 133 is smaller. When the included angle between the first extending portion 132a and the second extending portion 132b is greater, the buttons 133 are farther with each other, therefore, the distance D of buttons 133 is greater, but it is not limit this invention.

In the current embodiment, for example, the cable 130 includes three buttons (no shown), two of three buttons may be disposed on the first extending portion 132a and the other of three buttons may be disposed on the second extending portion 132b as needed (no shown).

In another alternative embodiment, two of three buttons may be disposed on the second extending portion 132b and the other of three buttons may be disposed on the first extending portion 132a as needed (no shown).

In another alternative embodiment, for example, the cable 130 further includes three buttons (no shown), the circuit board 132 further includes a first extending portion 132a, a second extending portion 132b and a third extending portion (no shown). At least one included angle between the first extending portion 132a, the second extending portion 132b and third extending portion (no shown), and the first extending portion 132a, the second extending portion 132b and third extending portion are corresponding to the first opening 131o1, respectively.

In the current embodiment, the back end device (no shown) may be controlled in accordance with the buttons 133, and a distance between the buttons 133 and the back end device is shorter than a distance between the handle 120 and the back end device. Therefore, the user controls the back end device in accordance with the buttons 133 fast and easily. And the included angle between the first extending portion 132a and the second extending portion 132b of the circuit board 132 to reduce the size and/or the volume of the cable 130. The user not only controls the buttons 133 of the cable 130 by single hand of the user but also controls the endoscope 100 simply, easily, fast and conveniently to achieve better operability and availability.

In the current embodiment, at least two second openings 134o2 are formed on the fixed portion 134. In this embodiment, each second opening 134o2 connects with two corresponding faces of the fixed portion 134, and each second opening 134o2 may be a hole. When the fixed portion 134 is disposed on the buttons 133, and located or disposed on the first opening 131o1 at the same, and the buttons 133 are further disposed through two second openings 134o2 of the fixed portion 134 and exposed from the second openings 134o2, respectively. Moreover, the surface area of the fixed portion 134 substantially may be equal to or smaller than the inside diameter or the size of the first opening 131o, and the shape of the fixed portion 134 may be equal to the shape of the first opening 131o1. Therefore, the fixed portion 134 maybe located at the first opening 131o1, and the buttons 133 and the circuit board 132 are fixed in the cable 130 in accordance with the fixed portion 134.

In the other current embodiment, the fixed portion 134 may cover on the first opening 131o1, in this embodiment, the surface area of the fixed portion 134 may be greater than the inside diameter or the size of the first opening 131o1, and the buttons 133 and the circuit board 132 are fixed in the cable 130 in accordance with the fixed portion 134.

In the current embodiment, the material of the housing 131, the buttons 133 or the fixed portion 134 may be a plastic material, an elastic material or a waterproof elastic material, but it is not limit this invention.

In the current embodiment, the cable 130 includes a fixed membrane (no shown), and the housing 131, the buttons 133 and the fixed portion 134 are covered with the fixed membrane. In other words, the housing 131 and the fixed portion 134 disposed on the housing 131 and the buttons 133 disposed through the fixed portion 134 are covered with the fixed membrane, and the fixed membrane may cover the whole of the housing 131, all buttons 133 and the fixed portion 134. The buttons 133 and the fixed portion 134 are not moved easily in accordance with the fixed membrane covers the cable 130. In this embodiment, the material of the fixed membrane may be a plastic material or a silicone material, further be a waterproof elastic material or a waterproof heat shrink material, but it is not limit this invention.

In another alternative embodiment, the cable 130 further includes a fixed membrane (no shown) , a fourth opening (no shown) is formed on the fixed membrane, and the fourth opening is corresponding to the first opening 131o1 of the housing 131. The fixed membrane cover on the housing 131 and the fixed portion 134, and the buttons 133 are exposed from the fourth opening. In other words, when the housing 131 and the fixed portion 134 disposed on the housing 131 are covered with the fixed membrane, the buttons 133 is exposed from the fourth opening without covered the fixed membrane. In another embodiment, the housing 131 is disposed through or covered with the fixed membrane, and the buttons 133 and the fixed portion 134 may be configured on the housing 131 through the fourth opening. In another embodiment, the housing 131 is disposed through or covered with the fixed membrane, and the buttons 133 may be disposed on the circuit board 132 and exposed from the fourth opening, and the fixed portion 134 may be configured on the housing 131 through the fourth opening to the buttons 133 are exposed from the second openings 13402 of the fixed portion 134.

In one embodiment, the fixed membrane may fix the housing 131, the buttons 133 and the fixed portion 134 in accordance with an ultrasonic processing. In other words, in the ultrasonic processing, the cable 130 with the fixed membrane is shook, and the fixed membrane may cover on the housing 131 of the cable 130 stably to fix the housing 131, the fixed portion 134 and the buttons 133.

Due to the fixed membrane not only may fix on the housing 131 of the cable 130 stably to make the housing 131, the fixed portion 134 and the buttons 133 to fix with each other but also the cable 130 may avoid water or dust enter therein in accordance with the fixed membrane when endoscope 100 are disassembled and the cable 130 is in the washing and/or sterilization processing to achieve the waterproof effect.

As shown in FIG. 1A, FIGS. 1B and 2, the handle 120 is disposed between the insert tube 110 and the cable 130, the handle 120 includes an operational unit 121 and a joystick 122, and the handle 120 may further include a controlling portion (no shown), the operational unit 121 and the joystick 122 are connected with each other, a controlling unit (no shown) is disposed in the controlling portion and connects to the insert tube 110; the user may control the joystick 122 to move the controlling portion with the controlling unit to control the insert tube 110 to bend any angle as needed. A connected hole 121o1 formed on the operational unit 121 may be configured for insertion of a surgical tool (not shown) to operate on various tissues.

The insert tube 110 includes at least one channel (no shown), for example, the insert tube 110 includes an instrument channel, a camera channel and a controlling channel (no shown). The controlling unit of the controlling portion is disposed through the controlling channel and connects to a front end of the insert tube 110; therefore, the insert tube 110 is bent and controlled in accordance with the joystick 122 of the handle 120. The instrument channel and the connected hole 121o1 of the operational unit 121 of the handle 120 are connected with each other, when at least one instrument insert from the connected hole 121o1 of the operational unit 121 of the handle 120, the instrument may expose from an opening of the instrument channel (no shown).

In one embodiment, the insert tube 110 are formed from a bending section 111 and extending section 112, the bending section 111 and the extending section 112 may be integrally formed in one piece or connected from two tubes, but it is not limit this invention. The bending section 111 connects to the distal section 140, and the extending section 112 connects to the handle 120. In this embodiment, the controlling unit further connects to the bending section 111, and the bending section 111 may be bent at any angle as needed in accordance with the controlling unit and the controlling portion controlled from the joystick 122.

The distal section 140 includes at least two holes (no shown), the holes connects to the instrument channel and the camera channel, respectively. The instrument maybe exposed from one hole of the distal section 140 to be operated. The endoscope 100 includes a camera module (no shown) . The camera module may be disposed in the other hole, and the camera module includes a camera, at least one light emitter unit, a first circuit board and a first wire (no shown). Wherein, the first circuit board electronically connects to the camera, the light emitter units and an end of the first wire. The camera and the light emitter units are disposed in the other hole. The other end of the first wire electronically connects to the circuit board 132 of the cable 130 through the handle 120. Therefore, the user may control or operate the camera module to capture the image from the camera and transfer the image to the connected portion 134 of the circuit board 132 of the cable 130 in accordance with the first circuit board of the camera module, and then, the image are transferred to the back end device in accordance with the transmitting line of the circuit board 132 of the cable 130 to display to show the human organs.

According to the buttons 133 are disposed on the cable 130, and the buttons 133 not only controls the elements of the front end of the endoscope 100, such as, the camera module and the bending section 111 of the insert tube 110 but also operates the back end device connected to the cable 130. Due to the distance between the cable 130 and the back end device is smaller than the distance between the handle 120 and the back end device, therefore, an operating signal of the buttons 133 of the cable 130 may be transferred to the back end device fast and easily. When the buttons 133 are broke, the broken cable 130 may be replaced by a new cable 130 to avoid repair the whole endoscope 100 and achieve good reliability and durability.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A cable comprising:
a housing having a space formed therein and a first opening connected to the space;
a circuit board received in the space and comprising a first extending portion, a second extending portion and an included angle between the first extending portion and the second extending portion;
at least two buttons disposed in the first extending portion and the second extending portion, respectively; and
a fixed portion disposed on each button and the first opening.

2. The cable of claim 1, wherein each button is exposed from the first opening, respectively.

3. The cable of claim 1, wherein the fixed portion has at least two second openings formed therein, and the buttons are disposed through and exposed from the second openings, respectively.

4. The cable of claim 1, wherein the fixed portion further covers on the first opening.

5. The cable of claim 1, wherein the housing further comprises a third opening formed on a face of one end of the housing, and the circuit board further comprises a connected portion disposed in the third opening.

6. The cable of claim 1, wherein the cable further comprises a fixed membrane, and the housing, the buttons and the fixed portion are covered with the fixed membrane.

7. The cable of claim 6, wherein the housing, the buttons and the fixed portion are fixed with the fixed membrane in accordance with an ultrasonic processing.

8. The cable of claim 1, wherein the cable further comprises a fixed membrane having a fourth opening, the housing and the fixed portion are covered with the fixed membrane, and the buttons are exposed from the fourth opening, respectively.

9. The cable of claim 8, wherein the housing and the fixed portion are fixed with the fixed membrane in accordance with an ultrasonic processing.

10. An endoscope comprising:
the cable claimed in claim 1;
an insert tube; and
a handle, one end of the handle connecting to one end of the cable, the other end of the handle connecting to one end of the insert tube.

11. An endoscope comprising:
an insert tube;
a cable comprising a housing, a circuit board, at least two buttons and a fixed portion, the housing having a space formed therein and a first opening connected to the space, the circuit board being received in the space and comprising a first extending portion, a second extending portion and an included angle between the first extending portion and the second extending portion, the buttons being disposed in the first extending portion and the second extending portion, respectively, and the fixed portion being disposed on each button and the first opening; and
a handle, one end of the handle connecting to one end of the cable, the other end of the handle connecting to one end of the insert tube.

12. The endoscope of claim 11, wherein each button is exposed from the first opening, respectively.

13. The endoscope of claim 11, wherein the fixed portion has at least two second openings formed therein, and the buttons are disposed through and exposed from the second openings, respectively.

14. The endoscope of claim 11, wherein the first opening is further covered with the fixed portion.

15. The endoscope of claim 11, wherein the housing further comprises a third opening formed on a face of one end of the housing, and the circuit board further comprises a connected portion disposed in the third opening.

16. The endoscope of claim 11, wherein the cable further comprises a fixed membrane, and the housing, the buttons and the fixed portion are covered with the fixed membrane.

17. The endoscope of claim 16, wherein the housing, the buttons and the fixed portion are fixed with the fixed membrane in accordance with an ultrasonic processing.

18. The endoscope of claim 11, wherein the cable further comprises a fixed membrane having a fourth opening, the housing and the fixed portion are covered with the fixed membrane, and the buttons are exposed from the fourth opening, respectively.

19. The endoscope of claim 18, wherein the housing and the fixed portion are fixed with the fixed membrane in accordance with an ultrasonic processing.
